(19) United States
(12) Patent Application Publication
(10) Pub. No.: US 2023/0301617 A1
(43) Pub. Date: Sep. 28, 2023

(54) STETHOSCOPE

(71) Applicants: ONKYO CORPORATION, Osaka (JP); KANAZAWA MEDICAL UNIVERSITY, Ishikawa (JP)

(72) Inventors: Makoto YOSHIDA, Osaka (JP); Kunimasa YAGI, Ishikawa (JP); Mikifumi SHIKIDA, Kochi (JP)

(21) Appl. No.: 18/125,760

(22) Filed: Mar. 24, 2023

(30) Foreign Application Priority Data
Mar. 28, 2022 (JP) 2022-051404

(51) Int. Cl. A61B 7/04; A61B 7/00

(52) U.S. Cl. CPC A61B 7/04; A61B 7/003

(57) ABSTRACT

A stethoscope system comprising: a plurality of sensors for collecting auscultatory sounds; and a controller which detects a first heart sound and a second heart sound of heart sounds based on a volume of the auscultatory sounds which are collected by the plurality of sensors.

### CROSS-REFERENCE TO RELATED APPLICATIONS

This application claims priority to Japanese Application No. 2022-051404, filed Mar. 28, 2022, the entire contents of which are incorporated herein by reference.

### FIELD

The present disclosure relates to a stethoscope.

### BACKGROUND

In stethoscopes, there is a so-called digital stethoscope which collects sounds such as heart sounds by a sensor such as a microphone, amplifies collected sounds and lets a doctor and the like listen to amplified sounds (for example, see JP2004-242849A). Herein, in the heart sounds, there are a first heart sound (S1) and a second heart sound (S2). The first heart sound is a sound which occurs when an atrium contracts, a blood fills a ventricle, an atrioventricular valve (a mitral valve and a tricuspid valve) closes and accordingly the atrium begins to contract. The second heart sound is a sound which occurs when the ventricle contract, the blood is sent to an artery, and an arterial valve (an aortic valve and a pulmonary valve) is closed. Conventionally, in the heart sounds, when it is judged whether it is the first heart sound or the second heart sound, it is necessary that the heart sounds and an electrocardiogram or the like are synchronized.

As described above, conventionally, in the heart sounds, there is a problem that it is necessary to synchronize the heart sounds and the electrocardiogram or the like when it is judged (detected) whether it is the first heart sound or the second heart sound. Further, there is a problem that the user of the stethoscope cannot judge whether the stethoscope is put on an appropriate position or not.

### SUMMARY OF THE DISCLOSURE

According to one aspect of the disclosure, there is provided a stethoscope system comprising: a plurality of sensors for collecting auscultatory sounds; and a controller which detects an first heart sound and an second heart sound of heart sounds based on a volume of the auscultatory sounds which are collected by the plurality of sensors.

### BRIEF DESCRIPTION OF THE DRAWINGS

FIG. 1 is a perspective diagram illustrating a stethoscope unit according to an embodiment of the present disclosure.

FIG. 2 is a top diagram illustrating the stethoscope unit according to an embodiment of the present disclosure.

FIG. 3(a) is a diagram illustrating auscultatory sounds which are collected by sensors.

FIG. 3(b) is an enlarged diagram of a part of FIG. 3(a).

FIG. 4 is a diagram illustrating the vicinity of a heart of a body.

FIG. 5 is a diagram of the stethoscope unit and a belt.

FIGS. 6(a), 6(b) and 6(c) are diagrams for describing a using state of the stethoscope unit.

FIG. 7 is a diagram illustrating one example of a display screen which displays a position where the stethoscope unit should be moved.

### DETAILED DESCRIPTION OF THE PREFERRED EMBODIMENTS

An objective of the present disclosure is to enable at least either of the following (1) and (2).

(1) To detect a first heart sound and a second heart sound in heart sounds without synchronizing with an electrocardiogram or the like.
(2) To inform a user of a stethoscope whether the stethoscope is put on an appropriate position or not.

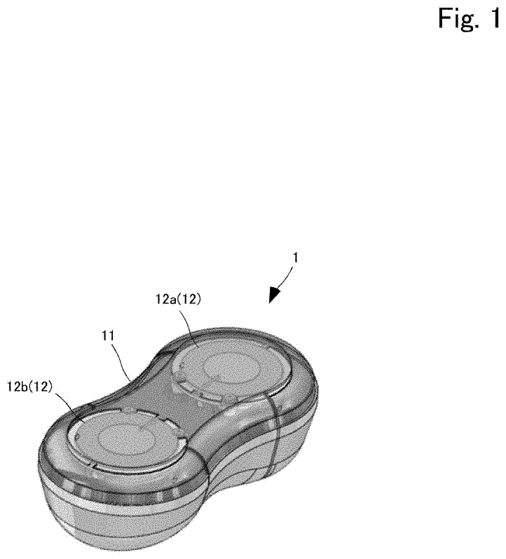

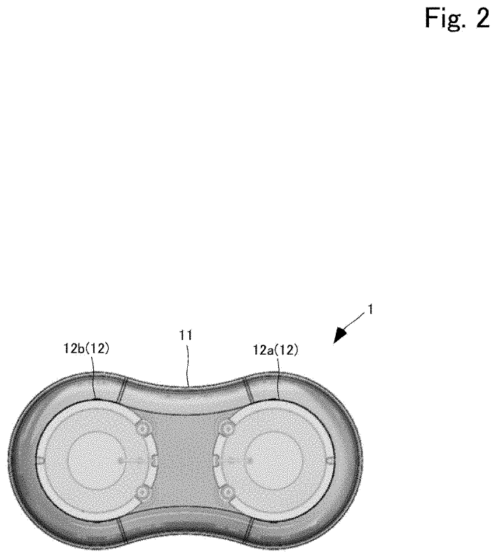

Hereinafter, an embodiment of the present disclosure will be described. FIG. 1 is a perspective diagram illustrating a stethoscope unit 1 according to an embodiment of the present disclosure. FIG. 2 is a top diagram illustrating the stethoscope unit 1 according to the embodiment of the present disclosure. The stethoscope unit 1 collects auscultatory sounds of a subject.

For example, the stethoscope unit 1 has an almost sandglass shape enclosure 11 when it is seen from a top. Each of sensors 12 for collecting the auscultatory sounds is housed within the enclosure 11. Namely, the enclosure 11 houses the sensors 12. In the present embodiment, the stethoscope unit 11 has two (a plurality of) sensors 12a and 12b. For example, each of the sensors 12 is a piezoelectric sensor which is consisted of a piezoelectric element and the like. The two sensors 12a and 12b are arranged side by side in a longitudinal direction of the enclosure 11.

In the present embodiment, for example, auscultatory sounds which are collected by the stethoscope unit 1 is sent to an external electronic device. For example, the electronic device is a smartphone, a personal computer (PC) or the like. The electronic device (controller) detects a first heart sound (S1) and a second heart sound (S2) of heart sounds based on a volume of the auscultatory sounds which are collected by the two (a plurality of) sensors 12a and 12b.

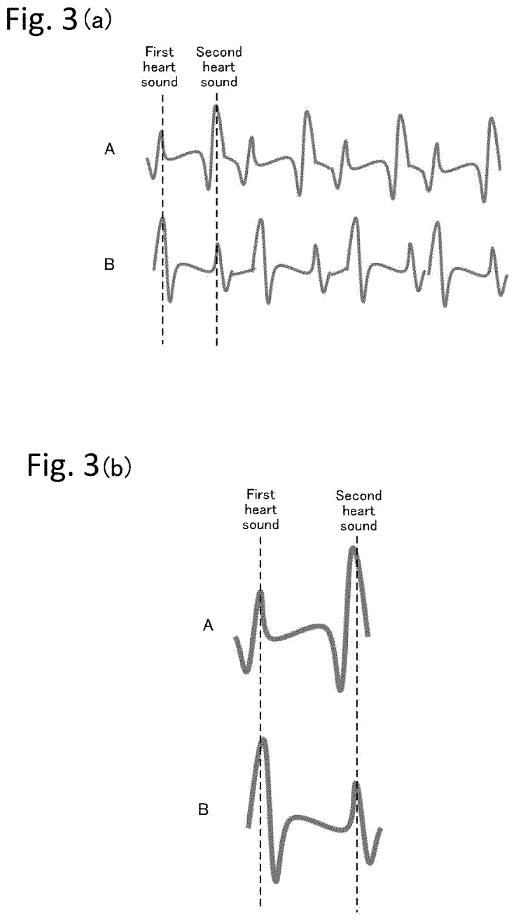

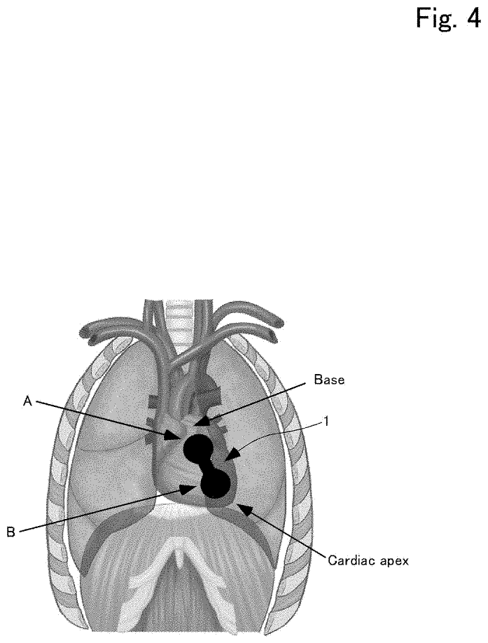

FIG. 3(a) is a diagram illustrating the auscultatory sounds which are collected by the sensors 12. FIG. 3(b) is an enlarged diagram of a part of FIG. 3(b). FIG. 4 is a diagram illustrating the vicinity of a heart of body. In FIG. 4, one sensor 12a positions near a base (at an auscultatory position A). Further, the other sensor 12b positions near a cardiac apex (at an auscultatory position B). The base is a height between second ribs, au upper end rear part and a place that a pulmonary artery and an aorta come out.

From a structure of the heart, when the first heart sound occurs, loud sounds (sounds of an aortic valve, etc.) occur near the base. Further, when the second heart sound occurs, loud sounds (sounds of a mitral valve, etc.) occur near the cardiac apex. Since a distance between the base and the cardiac apex is separate, the base is far from an occurring position of the second heart sound and the cardiac apex is far from an occurring position of the first heart sound. Thus, when the sounds are collected by the two sensors 12a and 12b near the base (at the auscultatory position A) and near the cardiac apex (at the auscultatory position B), a level difference of the auscultatory sounds always occurs.

Namely, as illustrated in FIG. 3, when the first heart sound occurs, the auscultatory sounds by the sensor 12a which positions near the base (at the auscultatory position A) are large and the auscultatory sound by the sensor 12b which positions near the cardiac apex (at the auscultatory position B) are small. Further, when the second heart sound occurs, the auscultatory sounds by the sensor 12a which positions near the base (at the auscultatory position A) are small and the auscultatory sounds by the sensor 12b which positions near the cardiac apex (at the auscultatory position B) are large. Thus, the electronic device can detect the first heart sound and the second heart sound of the heart sounds without synchronizing with an electrocardiogram or the like as a condition that the sensor 12a positions near the base (at the auscultatory position A) and the sensor 12b positions near the cardiac apex (at the auscultatory position B). Namely, the electronic device detects the first heart sound and the second heart sound of the heart sounds based on a volume of the auscultatory sounds which are collected by the two (a plurality of) sensors 12a and 12b and sensor position information indicating that either of sensors 12 positions near the base and either of sensors 12 positions near the cardiac apex. In FIG. 3, after the first heart sound occurs, the second heart sound occurs.

Further, the electronic device diagnoses based on the detected first heart sound and second heart sound of the heart sounds. As described in a homepage of Japanese Educational Clinical Cardiology Society (https://www.jeccs.org/medical/recommendation/bedside2/), if occurring timing of the first heart sound and second heart sound is known, it is easy to diagnose by means of a division of the second heart sound and the like. Further, the division of the second heart sound is most likely to be heard at auscultatory position A, which is closer to the cardiac base.

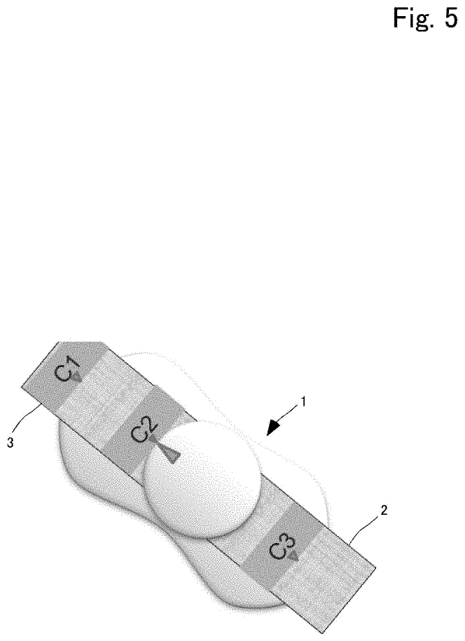

Herein, for example, as illustrated in FIG. 5, in the stethoscope unit 1, its auscultatory position can be fixed by a belt 2 (a fixing part) which is over a user's shoulder. Namely, the belt 2 fixes the stethoscope unit 1 on an auscultatory subject (a body).

Further, the electronic device detects a position of the stethoscope unit 1 based on a volume of auscultatory sounds which are collected by the two sensors 12a and 12b. Concretely, the electronic device detects the position of the stethoscope unit 1 based on a difference of the volume of the auscultatory sounds which are collected by the two sensors 12a and 12b. As described above, one sensor 12a positions near the base. Further, the other sensor 12b positions the cardiac apex. In this case, when the first heart sound occurs, the auscultatory sounds by the sensor 12a which positions near the base are large and the auscultatory sounds by the sensor 12b which positions near the cardiac apex are small. Further, when the second heart sound occurs, the auscultatory sounds by the sensor 12a which positions near the base are small and the auscultatory sounds by the sensor 12b which positions near the cardiac apex are large. Namely, since a volume difference of the heart sounds at the same time is clear, the electronic device can detect the position of the stethoscope unit 1 by using this.

The electronic device informs the detected position of the stethoscope unit 1. Further, the electronic device informs a position where the stethoscope should be moved based on the detected position of the stethoscope unit 1. For example, the electronic device informs by displaying on a display or the like. For example, the electronic device informs the position of the stethoscope unit 1 by displaying an image of the stethoscope unit 1 on an image of the body. In addition, the electronic device informs the position where the stethoscope unit 1 should be moved by indicating the position by an arrow or the like.

Herein, the electronic device detects the position of the stethoscope unit 1 based on a volume of the auscultatory sounds which are collected by the two sensors 12a and 12b at a plurality of positions. Further, the electronic device detects a position where the stethoscope unit 1 should be positioned based on the volume of the auscultatory sounds which are collected by the two sensors 12a and 12b at a plurality of positions. As described above, in FIG. 5, the belt 2 (a member) which fixes the stethoscope unit 1 on an auscultatory subject (a body) is illustrated. Marks 3 (C1 to C3 in FIG. 5) which become indicators when the user of the stethoscope moves the stethoscope unit 1 are illustrated on the belt 2.

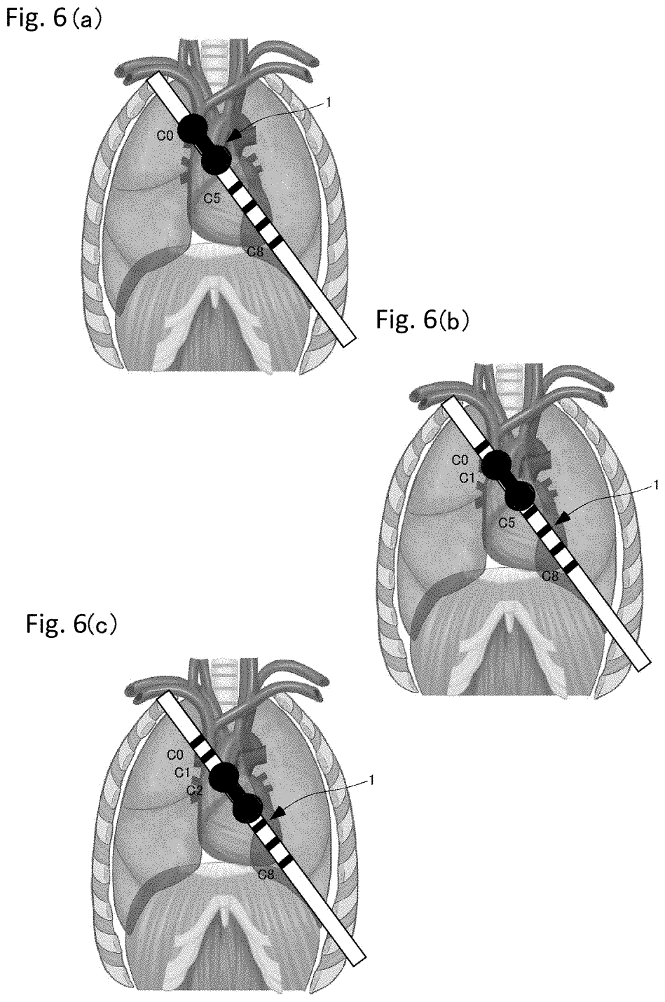

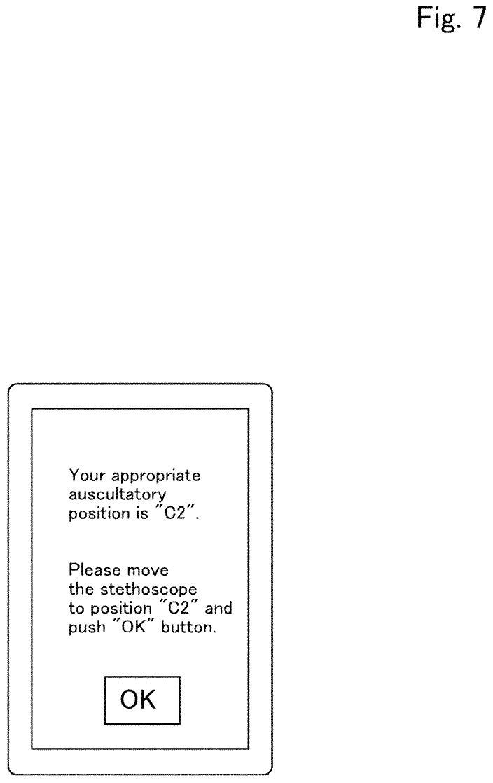

As illustrated in FIGS. 6(a)-6(c), the user moves the stethoscope unit 1 to C0 to C8 based on the marks 3 and lets the stethoscope unit 1 collect (record) the auscultatory sounds. In FIG. 6(a), the stethoscope unit 1 is on C0, in FIG. 6(b) the stethoscope unit 1 is on C1, and in FIG. 6(c), the stethoscope unit 1 is on C2. The electronic device detects a position where the stethoscope unit 1 should be positioned based on the volume of the auscultatory sounds which are collected by the two sensors 12a and 12b at the plurality of positions (herein, C0 to C8). And, for example, as illustrated in FIG. 7, the electronic device informs the position where the stethoscope unit 1 should be positioned. At this time, the electronic device displays a button for receiving confirmation whether the user lets the stethoscope unit 1 position at an informed position or not.

The electronic device detects whether the stethoscope unit 1 positions at the informed position based on the volume of the auscultatory sounds which are collected by the two sensors 12a and 12b. Like this, a calibration of a position of the stethoscope unit 1 is performed.

In the present embodiment, although the electronic device such as a smartphone, a PC or the like performs a detection of the first heart sound and the second heart sound of the heart sounds and a detection of the stethoscope unit 1, the stethoscope unit 1 may include a microcomputer or the like which controls the stethoscope unit 1 and the microcomputer may perform the above detection. In this case, the stethoscope is consisted of the stethoscope unit 1 which includes the microcomputer or the like as a stand-alone.

Further, in the present embodiment, although the sensors 12 are two, the sensors may be three or more.

As described above, in the present embodiment, the electronic device detects the first heart sound and the second heart sound of the heart sounds based on the volume of the auscultatory sounds which are collected by the two sensors 12a and 12b. Herein, in the heart sounds, generally, the first heart sound is heard loudly at the cardiac apex and the second heart sound is heard loudly at the base. Therefore, the first heart sound is collected loudly by the sensor 12 which positions near the cardiac apex and the second heart sound is collected loudly by the sensor 12 which positions near the base. Thus, the first heart sound and the second heart sound of the heart sounds can be detected without synchronizing with an electrocardiogram, or the like.

Further, in the present embodiment, the electronic device detects the position of the stethoscope unit 1 based on the volume of the auscultatory sounds which are collected by the two sensors 12a and 12b. Therefore, it can be informed to the user whether the stethoscope unit 1 is put on an appropriate position. Further, it can be informed to the user to move the stethoscope unit 1 to the appropriate position.

Further, in the present embodiment, the electronic device informs the detected position of the stethoscope unit 1. Thus, the user can grasp the position of the stethoscope unit.

Further, in the present embodiment, the electronic device informs the position where the stethoscope unit 1 should be moved based on the detected position of the stethoscope unit 1. This makes the user possible to grasp the position where the stethoscope unit 1 should be moved.

In the present embodiment, the belt 2 (the member) that the marks 3 which indicate the plurality of positions (for example, C0 to C8) are provided is used. For this reason, the user may move the stethoscope unit 1 based on the marks 3 and collect the auscultatory sound.

Further, in the present embodiment, the electronic device detects the position where the stethoscope unit 1 should be positioned based on the volume of the auscultatory sounds which are collected by the two sensors 12a and 12b. Therefore, the user can be informed to position the stethoscope unit 1 at the appropriate position, that is, the detected position where the stethoscope unit 1 should be positioned (see FIG. 7).

The embodiment of the present disclosure is described above, but the mode to which the present disclosure is applicable is not limited to the above embodiment and can be suitably varied without departing from the scope of the present disclosure.

The present disclosure can be suitably employed in a stethoscope.

What is claimed is:

1. A stethoscope system comprising:
a plurality of sensors for collecting auscultatory sounds; and
a controller which detects a first heart sound and a second heart sound of heart sounds based on a volume of the auscultatory sounds which are collected by the plurality of sensors.

2. The stethoscope system according to claim 1,
wherein the controller detects the first heart sound and the second heart sound of the heart sounds based on the volume of the auscultatory sounds which are collected by the plurality of sensors and sensor position information which indicates that either of the plurality of sensors positions near a base and either of the plurality of sensors positions near a cardiac apex.

3. The stethoscope system according to claim 1,
wherein the controller diagnoses based on the detected first heart sound and second heart sound of the heart sounds.

4. A stethoscope system comprising:
a stethoscope unit which has a plurality of sensors for collecting auscultatory sounds; and
a controller which detects a position of the stethoscope unit based on a volume of auscultatory sounds which are collected by the plurality of sensors.

5. The stethoscope system according to claim 4,
wherein the controller informs the detected position of the stethoscope unit.

6. The stethoscope system according claim 4,
wherein the controller informs a position where the stethoscope unit should be moved based on the detected position of the stethoscope unit.

7. The stethoscope system according to claim 4,
wherein the controller detects the position of the stethoscope unit based on a difference of the volume of the auscultatory sounds which are collected by the plurality of sensors.

8. The stethoscope system according to claim 4,
wherein the controller detects the position of the stethoscope unit based on the volume of the auscultatory sounds which are collected by the plurality of sensors at a plurality of positions.

9. The stethoscope system according to claim 8 further comprising:
a member at which marks indicating the plurality of positions are provided.

10. A stethoscope system comprising:
a stethoscope unit which has a plurality of sensors for collecting auscultatory sounds; and
a controller which detects a position where the stethoscope unit should be positioned based on a volume of the auscultatory sounds which are collected by the plurality of sensors.

11. The stethoscope system according to claim 1,
wherein the plurality of sensors are two sensors.

12. The stethoscope system according to claim 1 further comprising:
a fixing part which fixes the stethoscope unit on an auscultatory subject.

13. A stethoscope comprising:
a plurality of sensors for collecting auscultatory sounds; and
a controller which detects a first heart sound and a second heart sound of heart sounds based on a volume of the auscultatory sounds which are collected by the plurality of sensors.

14. A stethoscope comprising:
a plurality of sensors for collecting auscultatory sounds;
and a controller which detects a position of the stethoscope based on a volume of the auscultatory sounds which are collected by the plurality of sensors.
